(19) United States
(12) Patent Application Publication
(10) Pub. No.: US 2002/0146789 A1
(43) Pub. Date: Oct. 10, 2002

(54) Multi-domain proteinase inhibitor

(76) Inventors: Darrell C. Conklin, Seattle, WA (US); Zeren Gao, Redmond, WA (US)

Correspondence Address: Gary E. Parker, ZymoGenetics, Inc., 1201 Eastlake Avenue East, Seattle, WA, 98102, US

(21) Appl. No.: 09/819,136

(22) Filed: Mar. 27, 2001

Related U.S. Application Data
(60) Provisional application No. 60/193,642, filed on Mar. 31, 2000.

(51) Int. Cl. C12P 21/02; C12N 5/06; C12N 9/00; C07H 21/04

(52) U.S. Cl. 435/183; 435/320.1; 435/325; 435/69.1; 536/23.2

(57) ABSTRACT

A multidomain proteinase inhibitor and materials and methods for making it are disclosed. Fragments of the inhibitor are also disclosed. The proteinase inhibitor or fragments thereof may be used as components of cell culture media, in protein purification, and in certain therapeutic and diagnostic applications.

### CROSS-REFERENCE TO RELATED APPLICATION

This application claims the benefit of provisional application Serial No. 60/193,642, filed Mar. 31, 2000.

### BACKGROUND OF THE INVENTION

In animals, proteinases are important in wound healing, extracellular matrix destruction, tissue reorganization, and in cascades leading to blood coagulation, fibrinolysis, and complement activation. Proteinases are released by inflammatory cells for destruction of pathogens or foreign materials, and by normal and cancerous cells as they move through their surroundings.

The activity of proteinases is regulated by inhibitors; 10% of the proteins in blood serum are proteinase inhibitors (Roberts et al., Critical Reviews in Eukaryotic Gene Expression 5:385-436, 1995). One family of proteinase inhibitors, the Kunitz 20 inhibitors, includes inhibitors of trypsin, chymotrypsin, elastase, kallikrein, plasmin, coagulation factors XIa and IXa, and cathepsin G. These inhibitors thus regulate a variety of physiological processes, including blood coagulation, fibrinolysis, and inflammation.

Proteinase inhibitors regulate the proteolytic activity of target proteinases by occupying the active site and thereby preventing occupation by normal substrates. Although proteinase inhibitors fall into several unrelated structural classes, they all possess an exposed loop (variously termed an “inhibitor loop”, a “reactive core”, a “reactive site”, or a “binding loop”) which is stabilized by intermolecular interactions between residues flanking the binding loop and the protein core (Bode and Huber, Eur. J. Biochem. 204:433-451, 1992). Interaction between inhibitor and enzyme produces a stable complex which disassociates very slowly, releasing either virgin (uncleaved) inhibitor, or a modified inhibitor that is cleaved at the scissile bond of the binding loop.

The Kunitz inhibitors are generally basic, low molecular weight proteins comprising one or more inhibitory domains (“Kunitz domains”). The Kunitz domain is a folding domain of approximately 50-60 residues which forms a central anti-parallel beta sheet and a short C-terminal helix. This characteristic domain comprises six cysteine residues that form three disulfide bonds, resulting in a double-loop structure. Between the N-terminal region and the first beta strand resides the active inhibitory binding loop. This binding loop is disulfide bonded through the P2 Cys residue to the hairpin loop formed between the last two beta strands. Isolated Kunitz domains from a 5 variety of proteinase inhibitors have been shown to have inhibitory activity (e.g., Petersen et al., Eur. J. Biochem. 125:310-316, 1996; Wagner et al., Biochem. Biophys. Res. Comm. 186:1138-1145, 1992; Dennis et al., J. Biol. Chem. 270:25411-25417, 1995).

Proteinase inhibitors comprising one or more Kunitz domains include tissue factor pathway inhibitor (TFPI), tissue factor pathway inhibitor 2 (TFPI-2), amyloid β-protein precursor (AβPP), aprotinin, and placental bikunin. TFPI, an extrinsic pathway inhibitor and a natural anticoagulant, contains three tandemly linked Kunitz inhibitor domains. The amino-terminal Kunitz domain inhibits factor VIIa, plasmin, and cathepsin G; the second domain inhibits factor Xa, trypsin, and chymotrypsin; and the third domain has no known activity (Petersen et al., ibid.). TFPI-2 has been shown to be an inhibitor of the antidolytic and proteolytic activities of human factor Vila-tissue factor complex, factor Xia, plasma kallikrein, and plasmin (Sprecher et al., Proc. Natl. Acad. Sci. USA 91:3353-3357, 1994; Petersen et al., Biochem. 35:266-272, 1996). The ability of TFPI-2 to inhibit the factor VIIa-tissue factor complex and its relatively high levels of transcription in umbilical vein endothelial cells, placenta and liver suggests a specialized role for this protein in hemostasis (Sprecher et al., ibid.). Aprotinin (bovine pancreatic trypsin inhibitor) is a broad spectrum Kunitz-type serine proteinase inhibitor that has been shown to prevent activation of the clotting cascade. Aprotinin is a moderate inhibitor of plasma kallikrein and plamin, and blockage of fibrinolysis and extracorporeal coagulation have been detected in patients given aprotinin during open heart surgery (Davis and Whittington, Drugs 49:954-983, 1995; Dietrich et al., Thorac. Cardiovasc. Surg. 37:92-98, 1989). Aprotinin has also been used in the treatment of septic shock, adult respiratory distress syndrome, acute pancreatitis, hemorrhagic shock, and other conditions (Westaby, Ann. Thorac. Surg. 55:1033-1041, 1993; Wachtfogel et al., J. Thorac. Cardiovasc. Surg. 106:1-10, 1993). The clinical utility of aprotinin is believed to arise from its inhibitory activity towards plasma kallikrein or plasmin (Dennis et al., ibid.). Placental bikunin is a serine proteinase inhibitor containing two Kunitz domains (Delaria et al., J. Biol. Chem. 272:12209-12214, 1997). Individual Kunitz domains of bikunin have been expressed and shown to be potent inhibitors of trypsin, chymotrypsin, plasmin, factor XIa, and tissue and plasma kallikrein (Delaria et al., ibid.).

Known Kunitz-type inhibitors lack specificity and may have low potency. Lack of specificity can result in undesirable side effects, such as nephrotoxicity that occurs after repeated injections of high doses of aprotinin. These limitations may be overcome by preparing isolated Kunitz domains, which may have fewer side effects than traditional anticoagulants. Hence, there is a need in the art for additional Kunitz-type proteinase inhibitors.

### DESCRIPTION OF THE INVENTION

Within one aspect of the invention there is provided an isolated protein comprising a portion of SEQ ID NO:2, wherein the portion is selected from the group consisting of residues 33-75, residues 93-157, residues 203-286, residues 299-351, and residues 412-548. Within one embodiment, the protein is from 43 to 1600 amino acid residues in length. Within other embodiments, the protein comprises residues 299-409, residues 33-548, or residues 20-548 of SEQ ID NO:2. Within another embodiment, the protein further comprises an affinity tag. Exemplary affinity tags include, without limitation, maltose binding protein, polyhistidine, and Glu-Tyr-Met-Pro-Met-Glu (SEQ ID NO:4).

Within a second aspect of the invention there is provided an isolated protein comprising a portion of SEQ ID NO:2, wherein said portion is selected from the group consisting of residues 93-157, residues 203-286, residues 299-351, and residues 412-548.

Within a third aspect of the invention there is provided an isolated 25 polypeptide comprising at least 15 contiguous amino acid residues of SEQ ID NO:2, wherein the at least 15 contguous residues comprise residues 117-122, 525-530, 283-288, or 50-55 of SEQ ID NO:2.

Within a fourth aspect of the invention there is provided an expression vector comprising the following operably linked elements: (a) a transcription promoter; (b) a DNA segment encoding a protein comprising a portion of SEQ ID NO:2, wherein the portion is selected from the group consisting of residues 33-75, residues 93-157, residues 203-286, residues 299-351, and residues 412-548; and (c) a transcription terminator. Within one embodiment, the expression vector further comprises a secretory signal sequence operably linked to the DNA segment. Within a related embodiment, the secretory signal sequence encodes residues 1-19 of SEQ ID NO:2.

Within other embodiments, the protein comprises residues 299-409, residues 33-548, or residues 20-548 of SEQ ID NO:2. Within another embodiment, the vector further comprises a second DNA segment encoding an affinity tag as disclosed above operably linked to the DNA segment encoding the protein.

Within a fifth aspect of the invention there is provided an expression vector comprising the following operably linked elements: (a) a transcription promoter; (b) a DNA segment encoding a protein comprising a portion of SEQ ID NO:2, wherein the portion is selected from the group consisting of residues 93-157, residues 203-286, residues 299-351, and residues 412-548; and (c) a transcription terminator.

Within a sixth aspect of the invention there is provided a cultured cell containing an expression vector as disclosed above, wherein the cell expresses the DNA segment.

Within a seventh aspect of the invention there is provided a method of making a protein comprising the steps of culturing a cell as disclosed above under conditions whereby the DNA segment is expressed, and recovering the protein encoded by the DNA segment. Within one embodiment the expression vector further comprises a secretory signal sequence operably linked to the DNA segment, and the protein is secreted into and recovered from a culture medium in which the cell is cultured.

Within an eighth aspect of the invention there is provided a protein produced by the method disclosed above.

Within a ninth aspect of the invention there is provided an antibody that specifically binds to a protein as disclosed above.

These and other aspects of the invention will become evident upon reference to the following detailed description and the attached drawings.

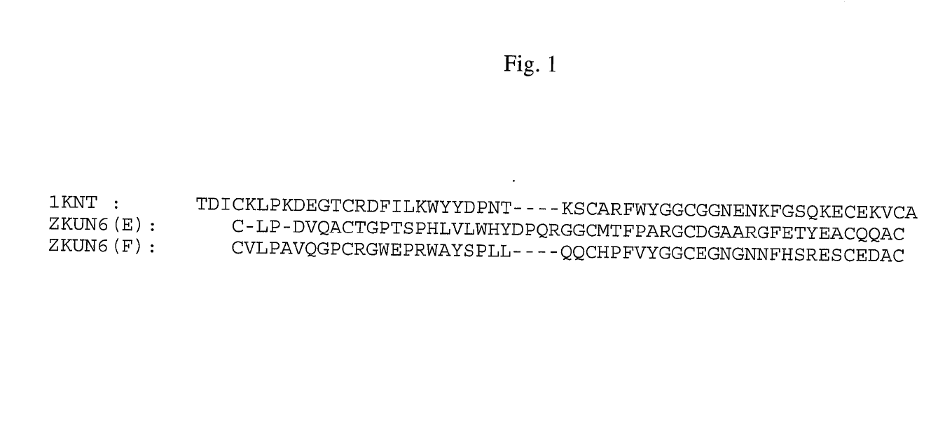

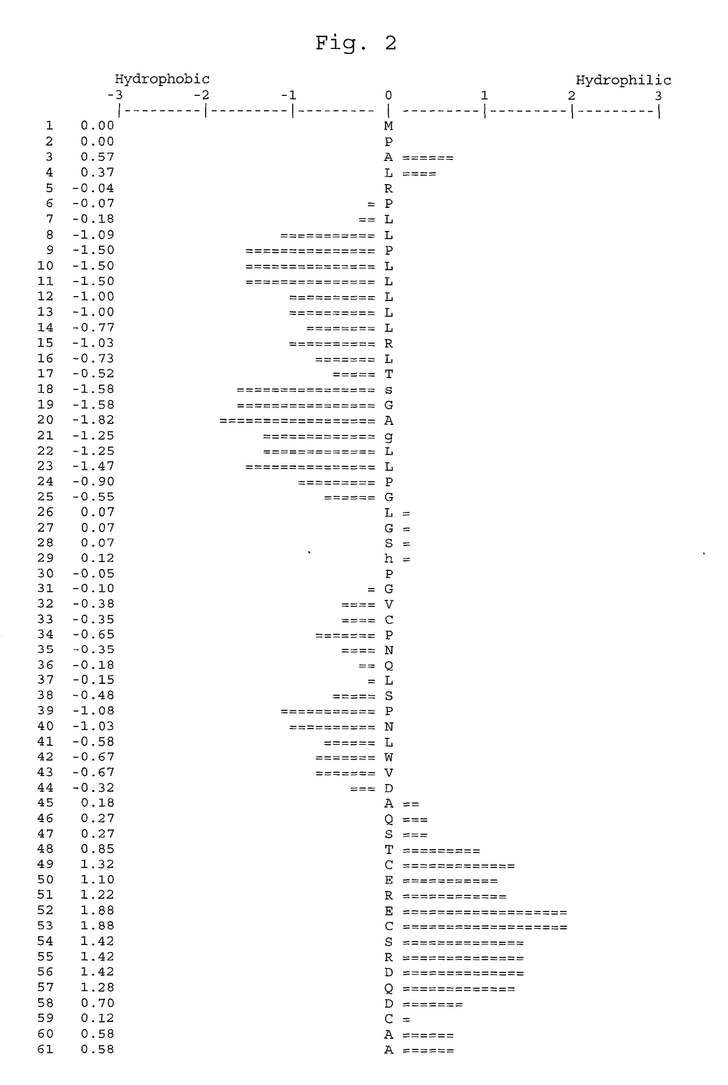

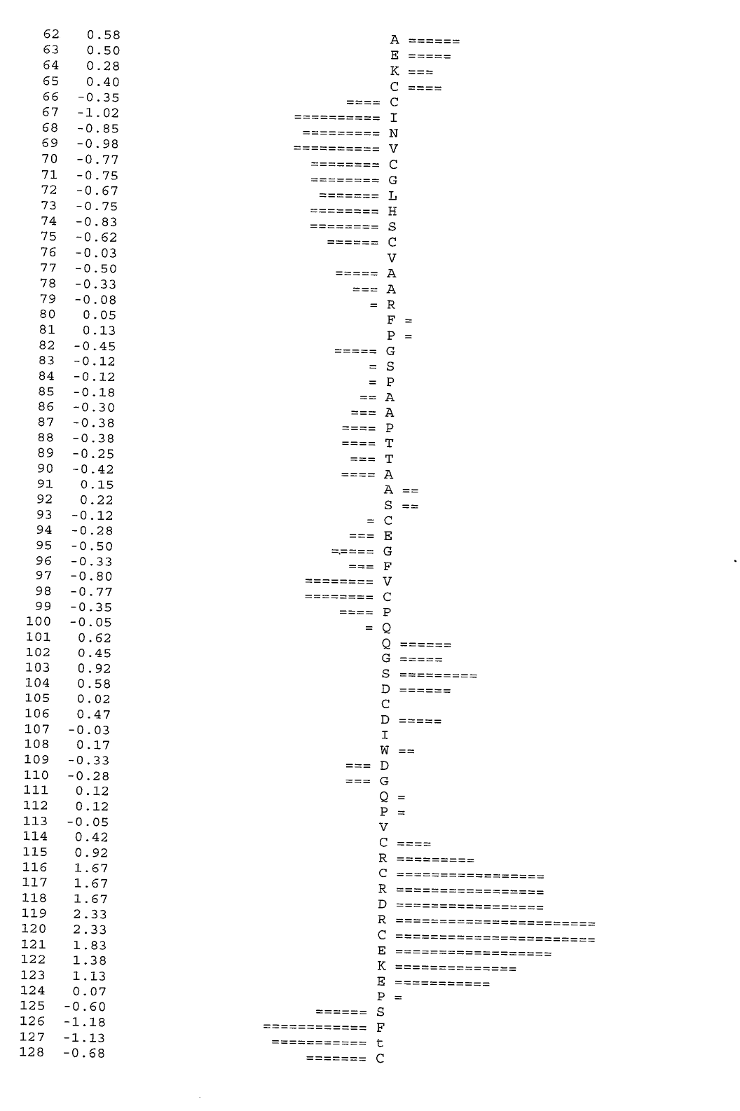

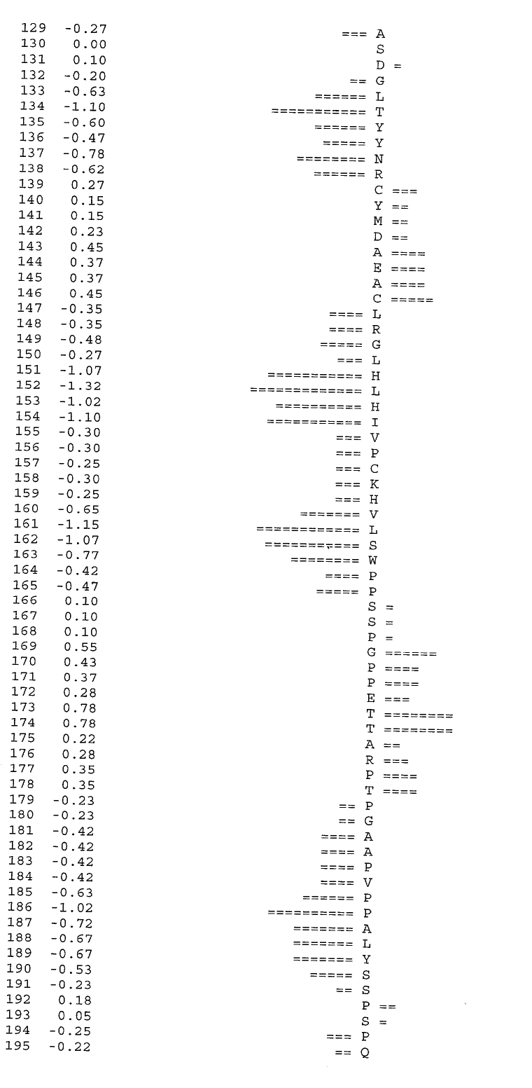

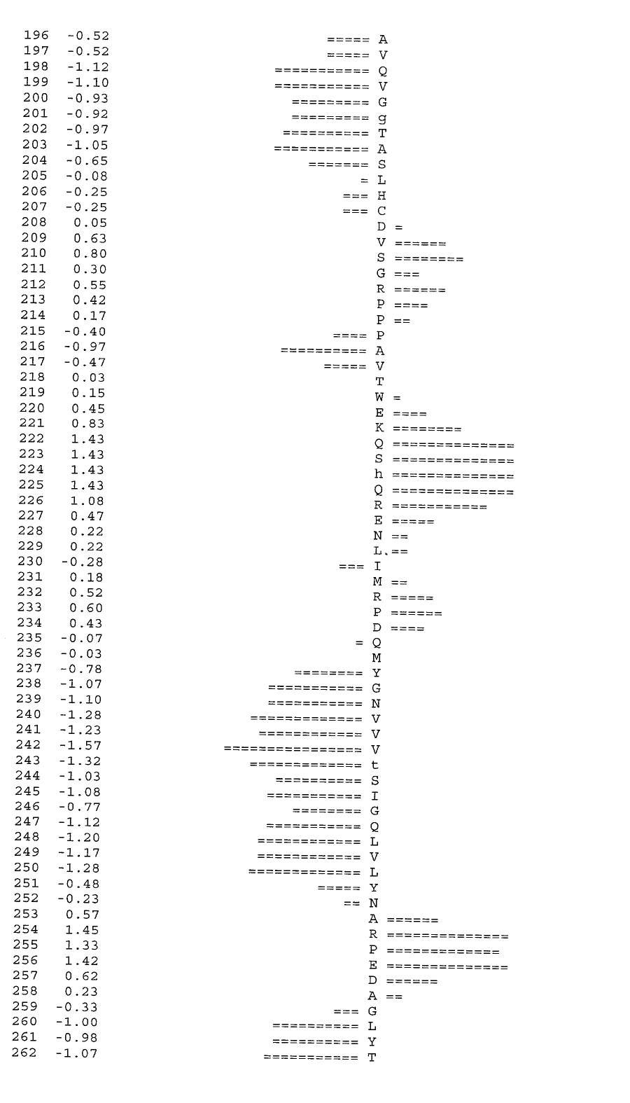

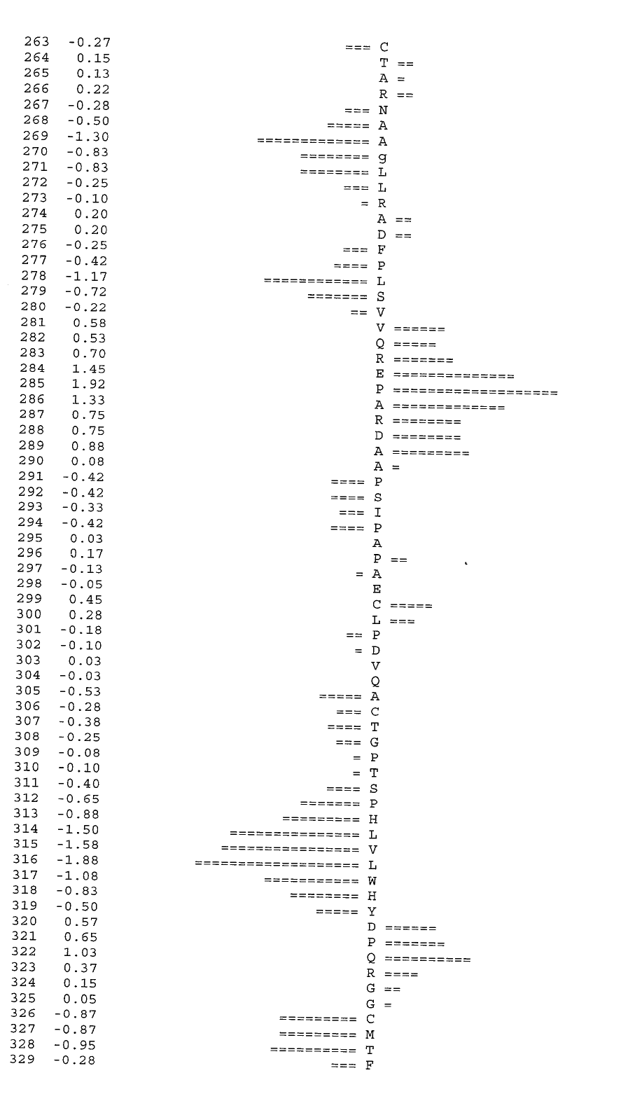

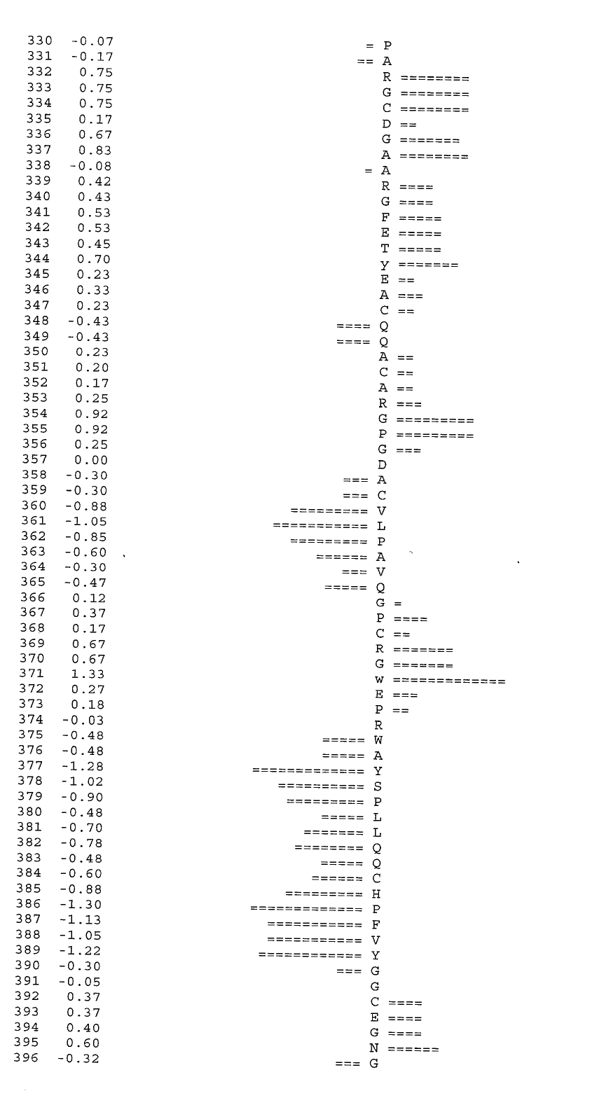

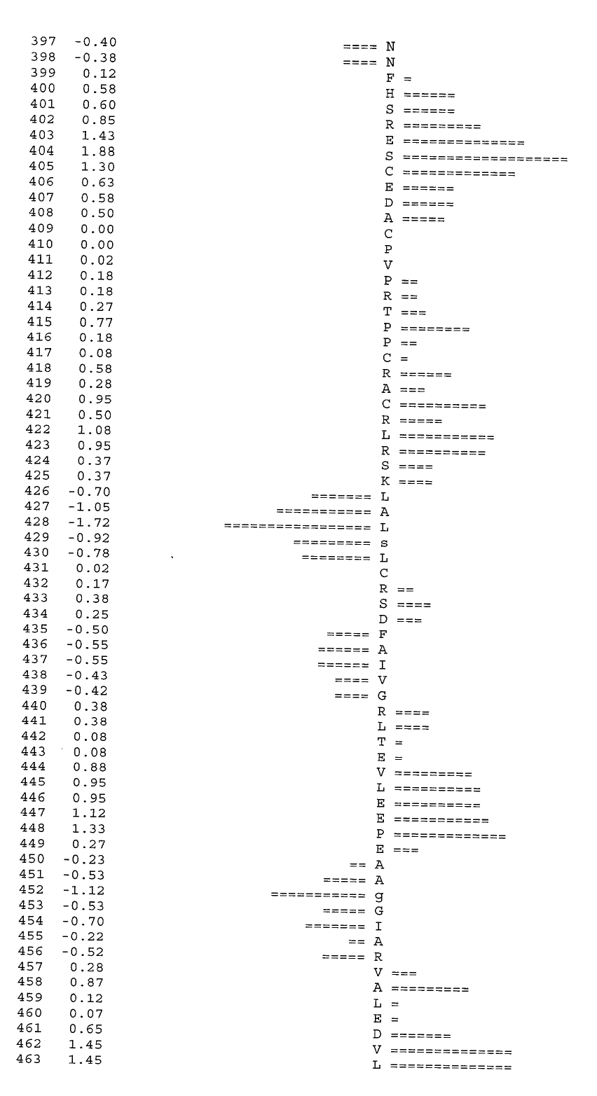

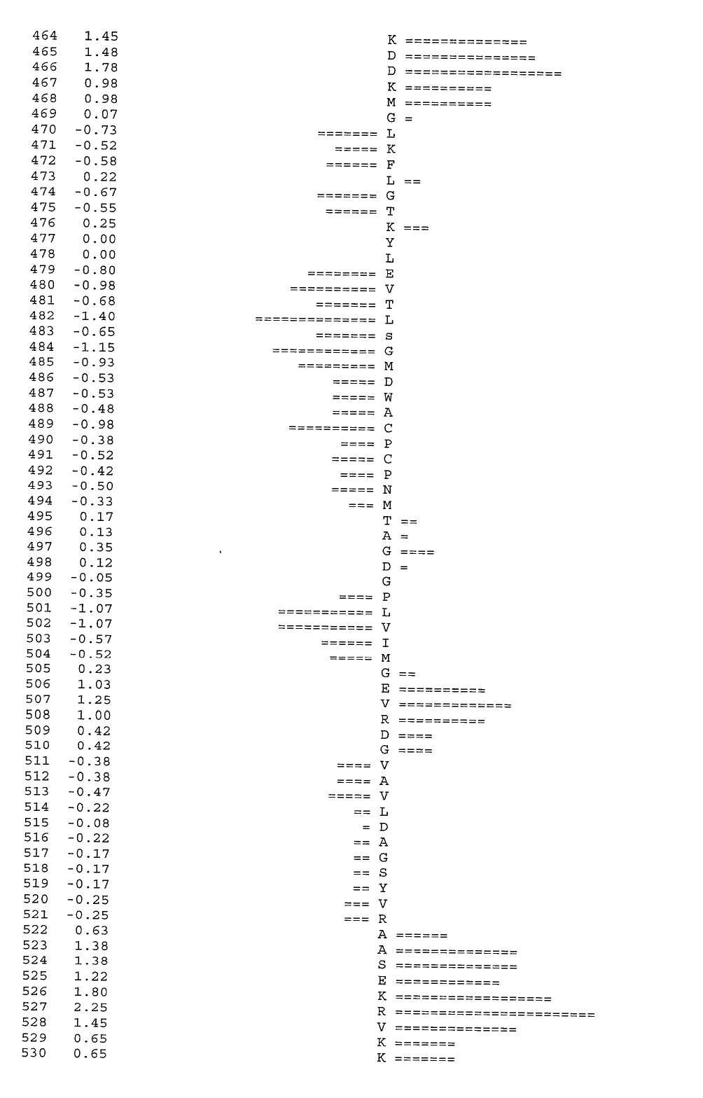

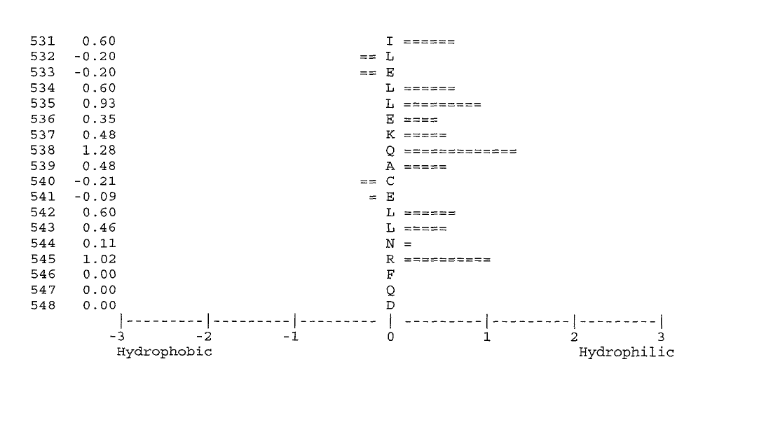

Within the drawings, FIG. 1 is an alignment of domains E and F of the protein shown in SEQ ID NO:2 with the Kunitz domain of human alpha 3 type VI collagen (“1KNT”; SEQ ID NO:3). FIG. 2 is a Hopp/Woods hydrophilicity profile of the amino acid sequence shown in SEQ ID NO:2. The profile is based on a sliding six-residue window. Buried G, S, and T residues and exposed H, Y, and W residues were ignored. These residues are indicated in the figure by lower case letters. Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any polypeptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Glu-Tyr-Met-Pro-Met-Glu; SEQ ID NO:4) (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Amersham Pharmacia Biotech, Piscataway, N.J.). 10 The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “complement” of a polynucleotide molecule is a polynucleotide molecule having a complementary base sequence and reverse orientation as compared to a reference sequence. For example, the sequence 5′ ATGCACGGG 3′ is complementary to 5′ CCCGTGCAT 3′.

“Conservative amino acid substitutions” are defined by the BLOSUM62 scoring matrix of Henikoff and Henikoff, Proc. Natl. Acad. Sci. USA 89:10915-10919, 1992. As used herein, the term “conservative amino acid substitution” refers to a 30 substitution represented by a BLOSUM62 value of greater than -1. For example, an amino acid substitution is conservative if the substitution is characterized by a BLOSUM62 value of 0, 1, 2, or 3. Preferred conservative amino acid substitutions are characterized by a BLOSUM62 value of at least one 1 (e.g., 1, 2 or 3), while more preferred conservative amino acid substitutions are characterized by a BLOSUM62 35 value of at least 2 (e.g., 2 or 3).The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

A “domain” is a contiguous polypeptide segment whose structure and/or function can be characterized in isolation. More specifically, a domain has one or more of the following properties:

1. It may have a particular role in determining protein subcellular or extracellular location, as in a transmembrane domain or a secretory signal peptide.

2. It may have a three-dimensional structure that exists in isolation of (separate from) its containing protein. Such domains can be recognized by the lack of intramolecular contacts between the domain and its containing protein. Such domains include, for example, tyrosine kinase domains of cell surface receptors and Kunitz proteinase inhibitor domains.

3. A domain may exhibit biological activity in isolation of its containing protein.

A “DNA segment” is a portion of a larger DNA molecule having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polynucleotide, polypeptide, or protein obtained from one species that is the functional counterpart of a polynucleotide, polypeptide, or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When these terms are applied to double-stranded molecules they are used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based on the discovery of a novel protein having a plurality of proteinase inhibitor domains. A representative human amino acid sequence of this protein, which has been designated “zkun6,” is shown in SEQ ID NO:2. Referring to SEQ ID NO:2, analysis of zkun6 indicates the presence of the domains shown in Table 1. As will be appreciated by those skilled in the art, domain boundaries are approximate and may vary by +/− five amino acid residues.

TABLE 1

| Domain | Residues | Description |
|---|---|---|
| A | 1-19 | secretory peptide |
| B | 33-75 | four-disulfide-core proteinase inhibitor |
| C | 93-157 | follistatin-type proteinase inhibitor |
| D | 203-286 | I-set immunoglobulin domain |
| E | 299-351 | Kunitz proteinase inhibitor domain #1 |
| F | 359-409 | Kunitz proteinase inhibitor domain #2 |
| G | 412-548 | Netrin domain |

Domain A is a hydrophobic secretory peptide that allows the zkun6 protein to be exported from the cell. Following this domain is a predominantly hydrophilic, short linker domain that forms the amino terminus of the mature protein.

Domain B is predicted to fold into a four-disulfide-core, or Chelonianin-type, serine proteinase inhibitor domain. The Chelonianin family is characterized by a common structural motif that comprises two adjacent beta-hairpin motifs, each consisting of two antiparallel beta strands connected by a loop region. The secondary structure of this motif is depicted by beta-sheet topology K (Branden and Tooze, Introduction to Protein Structure, Garland Publishing, Inc., 1991, p. 28). The beta strands are linked by intra-chain hydrogen bonding and by a network of four disulfide bonds. These disulfide bonds stablize the structure of the proteinase inhibitor and render it less susceptible to degradation. In view of this structural feature, the Chelonianin family is referred to as the “four-disulfide core” family of proteinase inhibitors. This family includes human antileukoproteinase, human elafin, guinea pig caltrin-like protein, human kallman syndrome protein, sea turtle chelonianin, the mouse WDNM1 protein, human epididymal secretory protein E4, trout TOP-2, and C. elegans C08G9. Several of these family members contain several copies of this structural motif. The four disulfide pairings in the B domain of zkun6 are Cys33-Cys66, Cys49-Cys70, Cys53-Cys65, Cys49-Cys75.

Domain C is predicted to fold into a structure similar to that of the follistatin homology domain of SPARC (also known as BM-40 and osteonectin; see, Hohenester et al., EMBO J. 16:3778-3786, 1997). This domain includes a beta hairpin structure followed by a small hydrophobic core of alpha/beta structure. Based on the disulfide bonding pattern in SPARC, the disulfide pairings in zkun6 can be inferred as Cys93-Cys 105, Cys98-Cys 114, Cys 116-Cys 146, Cys120-Cys 139, and Cys 128-Cysl57. The follistatin homology domain has substantial sequence similarity to the Kazal family (Bode and Huber, Eur. J. Biochem. 204:433-451, 1992) of serine proteinase inhibitors. Based on analogy with the crystal structures for the proteinase inhibitors PEC-60 (PDB 1PCE) and ovomucoid (PDB 1OVO), the putative proteinase binding site in domain C of zkun6 comprises the residues Cys120 (P3), Glu121 (P2), Lys122 (P1), Glu123 (PI′), and Pro124 (P2′) of SEQ ID NO:2. The scissile bond of the binding loop will therefore reside between the P1 and P1′ residues Lys122 and Glu123.

The D domain is predicted to fold into a structure similar to that determined for the telokin peptide (Swiss-Prot KMLS_HUMAN, PDB 1TLK). The telokin peptide falls into the immunoglobulins class of proteins, which are beta proteins folding into a beta-sandwich like structure (Bork et al., J. Mol. Biol. 242:309-320, 1994). These immunoglobulin domains have two beta sheets comprising 3+4 beta strands. The telokin peptide has been subclassified as an “I” set immunoglobulin domain. In zkun6 there is a potential intra-domain D disulfide bond between Cys207 and Cys263. Other proteins with I set immunoglobulin domains include titin, vascular and neural cell adhesion molecules, and twitchin. Domain D may serve an attachment function, such as attachment to extracellular matrix.

Domains E and F are predicted to fold into Kunitz-type serine proteinase inhibitor domains. Kunitz domains are approximately 50-60 residues in length and are characterized by an amino acid motif comprising six cysteine residues and having the sequence C-X(6, 8)-C-X(15, 19)-C-X(7)-C-X(12)-C-X(3)-C (SEQ ID NO:5), wherein C is cysteine, X is any naturally occuring amino acid residue, and the numerals indicate the number of such variable residues (wherein n1, n2 indicates from n1 to n2 residues). The second cysteine residue is in the P2 position. The Kunitz domain forms a central anti-parallel beta sheet and a short C-terminal helix. The structure is stabilized by three disulfide bonds. Between the N-terminal region and the first beta strand resides the active inhibitory binding loop. This binding loop is disulfide bonded through the P2 Cys residue to the hairpin loop formed between the last two beta strands.

Domain E has a Thr residue in the PI position (residue 307), which may indicate an unusual inhibitor specificity. An alignment of Kunitz domains E and F and the collagen Kunitz domain (SEQ ID NO:3) (see FIG. 1) can be combined with a homology model of zkun6 based on the X-ray structure to predict the function of certain residues in zkun6. Referring to SEQ ID NO:2, disulfide bonds are predicted to be formed in domain E by paired cysteine residues Cys299 -Cys351; Cys3O6 -Cys334; and Cys326 -Cys347. Within the predicted protease binding loop, the P1 residue is at Thr307, P2 at Cys306, and P1′ at Gly308.

Domain F has 45% amino acid sequence identity with the 51-residue kunitz domain in human alpha 3 type VI collagen (shown in SEQ ID NO:3). The structure of the latter domain has been solved by X-ray crystallography and by NMR (Arnoux et al., J. Mol. Biol. 246:609-617, 1995; Sorensen et al., Biochemistry 5 36:10439-10450, 1997). Referring to SEQ ID NO:2, disulfide bonds are predicted to be formed by paired cysteine residues Cys359 -Cys409; Cys368-Cys392; and Cys384-Cys405. The protease binding loop (P3-P4′) is expected to comprise residues 367-373 of SEQ ID NO:2 (Pro-Cys-Arg-Gly-Trp-Glu-Pro), with the P1 residue at Arg369, the P2 Cys residue at position 368, and the P1′ residue at Gly370. The Arg residue in the P1 position indicates that this domain should provide classic serine proteinase inhibitor activity.

Domain G shows homology to the C-terminal domains of netrins, complement proteins C3, C4, C5, secreted frizzled-related proteins, and procollagen C-proteinase enhancer proteins; and to the N-terninal domains of tissue inhibitors of metalloproteinases (TIMPs). This netrin-like domain, or “NTR module” (Banyai and Patthy, Protein Science 8:1636-1642, 1999), is characterized by the presence of six cysteine residues, which occur in zkun6 at residues 417, 420, 431, 489, 491, and 540 of SEQ ID NO:2. Disulfide bonds are predicted to be formed by paired cysteine residues 417-489, 420-491, and 431-540. Domain G has 27% amino acid sequence identity to 20 the C-terminal portion of a human Frzb protein (Hu et al., Biochem. Biophys. Res. Comm. 247:287-293, 1998. Netrin domains in other proteins have been associated with neuronal axon outgrowth activity, anti-apoptotic activity, and binding (and possibly inhibition) of metalloproteinases.

Zkun6 is thus a secreted, soluble protein with a multi-domain structure indicative of a multi-functional, broad spectrum proteinase inhibitor. Amino acid substitions can be made within the zkun6 sequence so long as highly conserved amino acid residues are retained and the higher order structure is not disrupted. Sequence alignments with related molecules provide guidance for introducing amino acid sequence changes into zkun6. For example, it is preferred to make substitutions within the zkun6 Kunitz domains by reference to the sequences of other Kunitz domains and the motif shown in SEQ ID NO:5. Within the present invention up to 20% of the amino acid residues in any domain of zkun6 can be replaced with other amino acid residues. The invention thus provides zkun6 variant proteins that are at least 80%, at least 85%, at least 90%, at least 95%, and at least 98% identical to one of domains B, C, D, E, F, or G of zkun6.

Percent sequence identity is determined by conventional methods. See, for example, Altschul et al., Bull. Math. Bio. 48:603-616, 1986, and Henikoff and Henikoff, Proc. Natl. Acad. Sci. USA 89:10915-10919, 1992. Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 2 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 2

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 1 | 1 | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The level of identity between amino acid sequences can be determined using the “FASTA” similarity search algorithm disclosed by Pearson and Lipman (Proc. Natl. Acad. Sci. USA 85:2444, 1988) and by Pearson (Meth. Enzymol. 183:63, 1990). Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, 1990 (ibid.).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art.

Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-809, 1993; and Chung et al., Proc. Natl. Acad. Sci. USA 90:10145-10149, 1993). In a second method, translation is carried out in Xenopus oocytes by microinjection of mutated mRNA and chemically aminoacylated suppressor tRNAs (Turcatti et al., J. Biol. Chem. 271:19991-19998, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-7476, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

Additional polypeptides may be joined to the amino and/or carboxyl termini of a zkun6 polypeptide, including a full-length zkun6 polypeptide, an isolated zkun6 domain as shown in Table 1, or a zkun6 variant as disclosed above. Amino and carboxyl extensions of a zkun6 polypeptide will be selected so as not to destroy or mask the proteinase-inhibiting activity of the protein by, for example, burying the active domain within the interior of the protein. There is a consequent preference for shorter extensions, typically 10-15 residues in length, often not exceeding 8 residues in length, when the zkun6 polypeptide is an isolated domain and the extension(s) will not be removed prior to use. There is considerable latitude in the permissible sequence of these extensions, although it is preferred to avoid the addition of cysteine residues in close proximity to a proteinase domain. For example, a zkun6 protein can comprise residues 299-351 of SEQ ID NO:2 with amino- and carboxyl-terminal dipeptides, wherein the individual amino acid residues of the dipeptides are any amino acid residue except cysteine.

Other amino- and carboxyl-terminal extensions that can be included in the proteins of the present invention include, for example, an amino-terminal methionine residue, a small linker peptide of up to about 20-25 residues, or an affinity tag as disclosed above. A protein comprising such an extension may further comprise a polypeptide linker and/or a proteolytic cleavage site between the zkun6 portion and the affinity tag. Cleavage sites include thrombin cleavage sites and factor Xa cleavage sites. For example, a zkun6 polypeptide of 529 amino acid residues can be expressed as a fusion comprising, from amino terminus to carboxyl terminus: maltose binding protein (approximately 370 residues)—polyhistidine (6 residues)—thrombin cleavage site (Leu-Val-Pro-Arg; SEQ ID NO:6)—zkun6, resulting in a polypeptide of approximately 909 residues. In a second example, a zkun6 polypeptide of 529 residues can be fused to E. coli β-galactosidase (1,021 residues; see Casadaban et al., J. Bacteriol. 143:971-980, 1980), a 10-residue spacer, and a 4-residue factor Xa cleavage site to yield a polypeptide of 1564 residues. Linker peptides and affinity tags provide for additional functions, such as binding to substrates, antibodies, binding proteins, and the like, and facilitate purification, detection, and delivery of zkun6 proteins. Within certain embodiments of the invention, a zkun6 polypeptide is prepared as a fusion protein to facilitate purification, and the fusion is subsequently cleaved to release the zkun6 portion. In another example, a zkun6 polypeptide (e.g., Kunitz domain) can be expressed as a secreted protein comprising a carboxyl-terminal receptor transmembrane domain, permitting the zkun6 polypeptide to be displayed on the surface of a cell. To span the lipid bilayer of the cell membrane, a minimum of about 20 amino acids are required in the transmembrane domain; these should predominantly be hydrophobic amino acids. The zkun6 polypeptide can be separated from the transmembrane domain by a spacer polypeptide, and can be contained within an extended polypeptide comprising a carboxyl-terminal transmembrane domain—spacer polypeptide—zkun6—amino-terminal polypeptide. Many receptor transmembrane domains and polynucleotides encoding them are known in the art. The spacer polypeptide will generally be at least about 50 amino acid residues in length, up to 200-300 or more residues. The amino terminal polypeptide may be up to 300 or more residues in length. Domain D, for example, may be prepared as a fusion protein wherein domain D provides a targetting or attachment function. Fusion proteins will generally be up to about 1600 amino acid residues in length, commonly up to about 1200 residues, and often shorter (e.g., 1000 or 750 residues).

Also disclosed herein are polynucleotide molecules, including DNA and RNA molecules, encoding zkun6 proteins. These polynucleotides include the sense strand; the anti-sense strand; and the DNA as double-stranded, having both the sense and anti-sense strand hydrogen bonded together. A representative DNA sequence encoding a human zkun6 protein is set forth in SEQ ID NO:1. DNA sequences encoding other zkun6 proteins can be readily generated by those of ordinary skill in the art based on the genetic code. Counterpart RNA sequences can be generated by substitution of U for T. Polynucleotides encoding zkun6 proteins and complementary polynucleotides are useful in the production of zkun6 proteins and for diagnostic and investigatory purposes.

Those skilled in the art will readily recognize that, in view of the degeneracy of the genetic code, considerable sequence variation is possible among these polynucleotide molecules. SEQ ID NO:7 is a degenerate DNA sequence that encompasses all DNAs that encode the zkun6 polypeptide of SEQ ID NO:2. Those skilled in the art will recognize that the degenerate sequence of SEQ ID NO:7 also provides all RNA sequences encoding SEQ ID NO:2 by substituting U for T. Thus, zkun6 polypeptide-encoding polynucleotides comprising nucleotide 1 to nucleotide 177 of SEQ ID NO:7 and their respective RNA equivalents are contemplated by the present invention. Table 3 sets forth the one-letter codes used within SEQ ID NO:7 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 3

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:7, encompassing all possible codons for a given amino acid, are set forth in Table 4.

TABLE 4

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | B | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TACTAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences shown in SEQ ID NO:2. Variant sequences can be readily tested for functionality as described herein.

One of ordinary skill in the art will also appreciate that different species can exhibit preferential codon usage. See, in general, Grantham et al., Nuc. Acids Res. 8:1893-1912, 1980; Haas et al. Curr. Biol. 6:315-324, 1996; Wain-Hobson et al., Gene 13:355-364, 1981; Grosjean and Fiers, Gene 18:199-209, 1982; Holm, Nuc. Acids Res. 14:3075-3087, 1986; and Ikemura, J. Mol. Biol. 158:573-597, 1982. “Preferential codon usage” is a term of art referring to the bias in codon usage within the genomes of certain species, whereby certain protein translation codons are more frequently used, thus favoring one or a few representatives of the possible codons encoding each amino acid (see Table 4). For example, the amino acid threonine (Thr) may be encoded by ACA, ACC, ACG, or ACT, but in mammalian cells ACC is the most commonly used codon. In other species, for example, insect cells, yeast, viruses or bacteria, different Thr codons may be preferred. Preferred codons for a particular species can be introduced into the polynucleotides of the present invention by a variety of methods known in the art. Introduction of preferred codon sequences into recombinant DNA can, for example, enhance production of the protein by making protein translation more efficient within a particular cell type or species. Therefore, the degenerate codon sequence disclosed in SEQ ID NO:7 serves as a template for optimizing expression of polynucleotides in various cell types and species commonly used in the art and disclosed herein. Sequences containing preferred codons can be tested and optimized for expression in various host cell species, and tested for functionality as disclosed herein.

It is preferred that zkun6 polynucleotides hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is up to about 0.03 M at pH 7 and the temperature is at least about 60° C.

As previously noted, zkun6-encoding polynucleotides include DNA and RNA. Methods for preparing DNA and RNA are well known in the art. In general, RNA is isolated from a tissue or cell that produces large amounts of zkun6 RNA. Such tissues and cells are identified by conventional procedures, such as Northern blotting (Thomas, Proc. Natl. Acad. Sci. USA 77:5201, 1980) or polymerase chain reaction (“PCR”) (Mullis, U.S. Pat. No. 4,683,202). Total RNA can be prepared using guanidine-HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. A zkun6-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zkun6 sequence disclosed herein. Within an additional method, the cDNA library can be used to transform or transfect host cells, and expression of the cDNA of interest can be detected with an antibody to zkun6 polypeptide. Similar techniques can also be applied to the isolation of genomic clones. Polynucleotides encoding zkun6 polypeptides are then identified and isolated by, for example, hybridization or PCR.

For recombinant expression, complementary DNA (cDNA) clones are often preferred, although for some applications (e.g., expression in transgenic animals) it may be preferable to use a genomic clone, or to modify a cDNA clone to include at least one genomic intron.

The polynucleotides of the present invention can also be synthesized using automated equipment (“gene machines”). The current method of choice is the phosphoramidite method. If chemically synthesized, double-stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. Gene synthesis methods are well known in the art. See, for example, Glick and Pasternak, Molecular Biotechnology, Principles & Applications of Recombinant DNA, ASM Press, Washington, D.C., 1994; Itakura et al., Annu. Rev. Biochem. 53: 323-356, 1984; and Climie et al., Proc. Natl. Acad. Sci. USA 87:633-637, 1990.

The zkun6 polynucleotide sequences disclosed herein can be used to isolate counterpart polynucleotides from other species (orthologs). These orthologous polynucleotides can be used, inter alia, to prepare the respective orthologous proteins.

These other species include, but are not limited to mammalian, avian, amphibian, reptile, fish, insect and other vertebrate and invertebrate species. Of particular interest are zkun6 polynucleotides abd polypeptides from other mammalian species, including murine, porcine, ovine, bovine, canine, feline, equine, and other primate polypeptides.

Orthologs of human zkun6 can be cloned using information and compositions provided by the present invention in combination with conventional cloning techniques. For example, a cDNA can be cloned by conventional techniques using MRNA obtained from a tissue or cell type that expresses zkun6 as disclosed herein.

Those skilled in the art will recognize that the sequence disclosed in SEQ ID NO:1 represents a single allele of human zkun6 and that natural variation, including allelic variation and alternative splicing, is expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the proteinase inhibiting activity of zkun6, are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Zkun6 proteins, including variants of wild-type zkun6, are tested for activity in protease inhibition assays, a variety of which are known in the art. Suitable assays include those measuring inhibition of trypsin, chymotrypsin, plasmin, cathepsin G, human leukocyte elastase, acrosin, leech tryptase, factor VIIa, or matrix metalloproteinases. See, for example, Petersen et al., Eur. J. Biochem. 235:310-316, 1996. In a typical procedure, the inhibitory activity of a test compound is measured by incubating the test compound with the proteinase, then adding an appropriate substrate, typically a chromogenic peptide substrate. See, for example, Norris et al. (Biol. Chem. Hoppe-Seyler 371:37-42, 1990). Briefly, various concentrations of the inhibitor are incubated in the presence of trypsin, plasmin, and plasma kallikrein in a low-salt buffer at pH 7.4, 25° C. After 30 minutes, the residual enzymatic activity is measured by the addition of a chromogenic substrate (e.g., S2251 (D-Val-Leu-Lys-Nan) or S2302 (D-Pro-Phe-Arg-Nan), available from DiaPharma Group, West Chester, Ohio) and a 30-minute incubation. Inhibition of enzyme activity is indicated by a decrease in absorbance at 405 nm or fluorescence Em at 460 nm. From the results, the apparent inhibition constant Ki is calculated. The inhibition of coagulation factors (e.g., factor VIIa, factor Xa) can be measured using chromogenic substrates or in conventional coagulation assays (e.g., clotting time of normal human plasma; Dennis et al., ibid.). Assays for inhibition of elastase, trypsin, or chymotrypsin are preferred for assaying domain B activity. Assays for inhibition of trypsin, acrosin, or leech tryptase are preferred for assaying domain C activity. Assays for trypsin, factor VIIa, and the like are preferred for assaying activity of domains E and F. Assays for inhibition of matrix matalloproteinases (e.g., collagenase, stromelysin) are preferred for assaying activity of domain G. Inhibition of matrix metalloproteinase MMP-2 can be assayed in the pancreatic cancer cell line PANC-1 that has been stimulated with the phorbol ester PMA. Activation of MMP-2 is assayed by gel zymography or by measuring the invasive potential of PANC cells in a Matrigel assay. See, Zervos et al., J. Surg. Res. 84:162-167, 1999.

Zkun6 proteins can be tested in animal models of disease, particularly tumor models, models of fibrinolysis, and models of imbalance of hemostasis. Suitable models are known in the art. For example, inhibition of tumor metastasis can be assessed in mice into which cancerous cells or tumor tissue have been introduced by implantation or injection (e.g., Brown, Advan. Enzyme Regul. 35:293-301, 1995; Conway et al., Clin. Exp. Metastasis 14:115-124, 1996). Effects on fibrinolysis can be measured in a rat model wherein the enzyme batroxobin and radiolabeled fibrinogen are administered to test animals. Inhibition of fibrinogen activation by a test compound is seen as a reduction in the circulating level of the label as compared to animals not receiving the test compound. See, Lenfors and Gustafsson, Semin. Thromb. Hemost. 22:335-342, 1996. Zkun6 proteins can be delivered to test animals by injection or infusion, or can be produced in vivo by way of, for example, viral or naked DNA delivery systems or transgenic expression.

Exemplary viral delivery systems include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161-189, 1994; and Douglas and Curiel, Science & Medicine 4:44-53, 1997). The adenovirus system offers several advantages: adenovirus can (i) accommodate relatively large DNA inserts; (ii) be grown to high titer; (iii) infect a broad range of mammalian cell types; and (iv) be used with a large number of available vectors containing different promoters. Also, because adenoviruses are stable in the bloodstream, they can be administered by intravenous injection. By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene is deleted from the viral vector, and the virus will not replicate unless the E1 gene is provided by the host cell (e.g., the human 293 cell line). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a signal sequence is present, secrete) the heterologous protein. Secreted proteins will enter the circulation in the highly vascularized liver, and effects on the infected animal can be determined.

An alternative method of gene delivery comprises removing cells from the body and introducing a vector into the cells as a naked DNA plasmid. The transformed cells are then re-implanted in the body. Naked DNA vectors are introduced into host cells by methods known in the art, including transfection, electroporation, microinjection, transduction, cell fusion, DEAE dextran, calcium phosphate precipitation, use of a gene gun, or use of a DNA vector transporter. See, Wu et al., J. Biol. Chem. 263:14621-14624, 1988; Wu et al., J. Biol. Chem. 267:963-967, 1992; and Johnston and Tang, Meth. Cell Biol. 43:353-365, 1994.

Transgenic mice, engineered to express a zkun6 gene, and mice that exhibit a complete absence of zkun6 gene function, referred to as “knockout mice” (Snouwaert et al., Science 257:1083, 1992), can also be generated (Lowell et al., Nature 366:740-742, 1993). These mice are employed to study the zkun6 gene and 2 5 the encoded protein in an in vivo system. Transgenic mice are particularly useful for investigating the role of zkun6 proteins in early development because they allow the identification of developmental abnormalities or blocks resulting from the over- or underexpression of a specific factor.

The zkun6 polypeptides of the present invention, including full-length polypeptides, biologically active fragments, and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zkun6 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zkun6 polypeptide into the secretory pathway of a host cell, a secretory signal sequence (also known as a leader sequence, prepro sequence or pre sequence) is provided in the expression vector. The secretory signal sequence may be that of zkun6, or may be derived from another secreted protein (e.g., t-PA) or synthesized de novo. The secretory signal sequence is operably linked to the zkun6 DNA sequence, i.e., the two sequences are joined in the correct reading frame and positioned to direct the newly sythesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are suitable hosts for use within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, 10801 University Boulevard, Manassas, Va.. Suitable promoters include those from metallothionein genes (U.S. Pat. Nos. 4,579,821 and 4,601,978), SV-40, cytomegalovirus (U.S. Pat. No. 4,956,288), and the adenovirus major late promoter. Expression vectors for use in mammalian cells include pZP-1 and pZP-9, which have been deposited with the American Type Culture Collection, 10801 University Boulevard, Manassas, Va. under accession numbers 98669 and 98668, respectively, and derivatives thereof.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” An exemplary selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Insect cells can be infected with recombinant baculovirus vectors, which are commonly derived from Autographa californica multiple nuclear polyhedrosis virus (AcMNPV). DNA encoding the polypeptide of interest is inserted into the viral genome in place of the polyhedrin gene coding sequence by homologous recombination in cells infected with intact, wild-type AcMNPV and transfected with a transfer vector comprising the cloned gene operably linked to polyhedrin gene promoter, terminator, and flanking sequences. The resulting recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. For example, production of recombinant proteins in Pichia methanolica is disclosed in U.S. Pat. Nos. 5,716,808, 5,736,383, 25 5,854,039, and 5,888,768. See also, Gleeson et al., J. Gen. Microbiol. 132:3459-3465, 1986 and Cregg, U.S. Patent No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

Prokaryotic host cells, including strains of the bacteria Escherichia coli, Bacillus and other genera are also useful host cells within the present invention.

Techniques for transforming these hosts and expressing foreign DNA sequences cloned therein are well known in the art (see, e.g., Sambrook et al., ibid.). When expressing a zkun6 polypeptide in bacteria such as E. coli, the polypeptide may be retained in the cytoplasm or may be directed to the periplasmic space by a bacterial secretion sequence. In the former case, the cells are lysed, and the zkun6 polypeptide is recovered from the lysate. If the polypeptide is present in the cytoplasm as insoluble granules, the cells are lysed, and the granules are recovered and denatured using, for example, guanidine isothiocyanate or urea. The denatured polypeptide can then be refolded by diluting the denaturant, such as by dialysis against a solution of urea and a combination of reduced and oxidized glutathione, followed by dialysis against a buffered saline solution. In the alternative, the polypeptide may be recovered from the cytoplasm in soluble form and isolated without the use of denaturants. The polypeptide is recovered from the cell as an aqueous extract in, for example, phosphate buffered saline. To capture the zkun6 polypeptide, the extract is applied directly to a chromatographic medium, such as an immobilized antibody. Secreted polypeptides can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. Liquid cultures are provided with sufficient aeration by conventional means, such as shaking of small flasks or sparging of fermentors.

Depending on the intended use, the proteins of the present invention can be purified to ≧80% purity, to ≧90% purity, to ≧95% purity, or to a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

Zkun6 proteins are purified by conventional protein purification methods, typically by a combination of chromatographic techniques. Polypeptides comprising a polyhistidine affinity tag (typically about 6 histidine residues) are purified by affinity chromatography on a nickel chelate resin. See, for example, Houchuli et al., Bio/Technol. 6: 1321-1325, 1988.

Using methods known in the art, zkun6 proteins can be produced glycosylated or non-glycosylated; PEGylated or non-PEGylated; and may or may not include an initial methionine amino acid residue.

The zkun6 proteins are contemplated for use in the treatment or prevention of conditions associated with excessive proteinase activity, in particular an excess of trypsin, plasmin, kallikrein, elastase, cathepsin G, proteinase-3, thrombin, factor VIIa, factor IXa, factor Xa, factor XIa, factor XIIa, or matrix metalloproteinases. Such conditions include, but are not limited to, acute pancreatitis, cardiopulmonary bypass (CPB)-induced pulmonary injury, allergy-induced protease release, deep vein thrombosis, myocardial infarction, shock (including septic shock), hyperfibrinolytic hemorrhage, emphysema, rheumatoid arthritis, adult respiratory distress syndrome, chronic inflammatory bowel disease, psoriasis, and other inflammatory conditions. Zkun6 proteins are also contemplated for use in preservation of platelet function, organ preservation, and wound healing.

Zkun6 proteins may be useful in the treatment of conditions arising from an imbalance in hemostasis, including acquired coagulopathies, primary fibrinolysis and fibrinolysis due to cirrhosis, and complications from high-dose thrombolytic therapy. Acquired coagulopathies can result from liver disease, uremia, acute disseminated intravascular coagulation, post-cardiopulmonary bypass, massive transfusion, or Warfarin overdose (Humphries, Transfusion Medicine 1:1181-1201, 1994). A deficiency or dysfunction in any of the procoagulant mechanisms predisposes the patient to either spontaneous hemorrhage or excess blood loss associated with trauma or surgery. Acquired coagulopathies usually involve a combination of deficiencies, such as deficiencies of a plurality of coagulation factors, and/or platelet dysfunction. In addition, patients with liver disease commonly experience increased fibrinolysis due to an inability to maintain normal levels of α₂-antiplasmin and/or decreased hepatic clearance of plasminogen activators (Shuman, Hemorrhagic Disorders, in Bennet and Plum, eds. Cecil Textbook of Medicine, 20th ed., W.B. Saunders Co., 1996). Primary fibrinolysis results from a massive release of plasminogen activator. Conditions associated with primary fibrinolysis include carcinoma of the prostate, acute promyelocytic leukemia, hemangiomas, and sustained release of plasminogen activator by endothelial cells due to injection of venoms. The condition becomes critical when enough plasmin is activated to deplete the circulating level of α₂-antiplasmin (Shuman, ibid.). Data suggest that plasmin on endothelial cells may be related to the pathophysiology of bleeding or rethrombosis observed in patients undergoing high-dose thrombolytic therapy for thrombosis. Plasmin may cause further damage to the thrombogenic surface of blood vessels after thrombolysis, which may result in rethrombosis (Okajima, J. Lab. Clin. Med. 126:1377-1384, 1995).

Additional antithrombotic uses of zkun6 proteins include treatment or prevention of deep vein thrombosis, pulmonary embolism, and post-surgical thrombosis. Zkun6 proteins may also be used within methods for inhibiting blood coagulation in mammals, such as in the treatment of disseminated intravascular coagulation. Zkun6 proteins may thus be used in place of known anticoagulants such as heparin, coumarin, and anti-thrombin III. Such methods will generally include administration of the protein in an amount sufficient to produce a clinically significant inhibition of blood coagulation. Such amounts will vary with the nature of the condition to be treated, but can be predicted on the basis of known assays and experimental animal models, and will in general be within the ranges disclosed below.

Zkun6 proteins may also find therapeutic use in the blockage of proteolytic tissue degradation. Proteolysis of extracellular matrix, connective tissue, and other tissues and organs is an element of many diseases. This tissue destruction is beleived to be initiated when plasmin activates one or more matrix metalloproteinases (e.g., collagenase and metallo-elastases). Inhibition of plasmin by zkun6 proteins may thus be beneficial in the treatment of these conditions.

Matrix metalloproteinases (MMPs) are believed to play a role in metastases of cancers, abdominal aortic aneurysm, multiple sclerosis, rheumatoid arthritis, osteoarthritis, trauma and hemorrhagic shock, and corneal ulcers. MMPs produced by tumor cells break down and remodel tissue matrices during the process of metastatic spread. There is evidence to suggest that MMP inhibitors may block this activity (Brown, Advan. Enzyme Regul. 35:293-301, 1995). Abdominal aortic aneurysm is characterized by the degradation of extracellular matrix and loss of structural integrity of the aortic wall. Data suggest that plasmin may be important in the sequence of events leading to this destruction of aortic matrix (Jean-Claude et al., Surgery 116:472-478, 1994). Proteolytic enzymes are also believed to contribute to the inflammatory tissue damage of multiple sclerosis (Gijbels, J. Clin. Invest. 94:2177-2182, 1994). Rheumatoid arthritis is a chronic, systemic inflammatory disease predominantly affecting joints and other connective tissues, wherein proliferating inflammatory tissue (panus) may cause joint deformities and dysfunction (see, Arnett, in Cecil Textbook of Medicine, ibid.). Osteoarthritis is a chronic disease causing deterioration of the joint cartilage and other joint tissues and the formation of new bone (bone spurs) at the margins of the joints. There is evidence that MMPs participate in the degradation of collagen in the matrix of osteoarthritic articular cartilage. Inhition of MMPs results in the inhibition of the removal of collagen from cartilage matrix (Spirito, Inflam. Res. 44 (supp. 2):S131-S132, 1995; O'Byrne, Inflam. Res. 44 (supp. 2):S117-S118, 1995; Karran, Ann. Rheumatic Disease 54:662-669, 1995). Zkun6 proteins may also be useful in the treatment of trauma and hemorrhagic shock. Data suggest that administration of an MMP inhibitor after hemorrhage improves cardiovascular response, hepatocellular function, and microvascular blood flow in various organs (Wang, Shock 6:377-382, 1996). Corneal ulcers, which can result in blindness, manifest as a breakdown of the collagenous stromal tissue. Damage due to thermal or chemical injury to corneal surfaces often results in a chronic wound-healing situation. There is direct evidence for the role of MMPs in basement membrane defects associated with failure to re-epithelialize in cornea or skin (Fini, Am. J. Pathol. 149:1287-1302, 1996).

The zkun6 proteins of the present invention may be combined with other therapeutic agents to augment the activity (e.g., antithrombotic or anticoagulant activity) of such agents. For example, a zkun6 protein may be used in combination with tissue plasminogen activator in thrombolytic therapy.

Doses of zkun6 proteins will vary according to the severity of the condition being treated and may range from approximately 10 μg/kg to 10 mg/kg body weight, preferably 100 μg/kg to 5 mg/kg, more preferably 100 μg/kg to 1 mg/kg. The proteins are formulated in a pharmaceutically acceptable carrier or vehicle. It is preferred to prepare them in a form suitable for injection or infusion, such as by dilution with with sterile water, an isotonic saline or glucose solution, or similar vehicle. In the alternative, the protein may be packaged as a lyophilized powder, optionally in combination with a pre-measured diluent, and resuspended immediately prior to use. Pharmaceutical compositions may further include one or more excipients, preservatives, solubilizers, buffering agents, albumin to prevent protein loss on vial surfaces, etc. Formulation methods are within the level of ordinary skill in the art. See, Remington: The Science and Practice of Pharmacy, Gennaro, ed., Mack Publishing Co., Easton, Pa., 19th ed., 1995.

Gene therapy provides an alternative therapeutic approach for delivery of zkun6 proteins. If a mammal has a mutated or absent zkun6 gene, a polynucleotide encoding a zkun6 protein can be introduced into the cells of the mammal. In one embodiment, a gene encoding a zkun6 protein is introduced in vivo in a viral vector. Such vectors include an attenuated or defective DNA virus, such as herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, without limitation, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

Within another embodiment, a zkun6 polynucleotide can be introduced in a retroviral vector, as described, for example, by Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication No. WO 95/07358; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natil. Acad. Sci. USA 85:8027-31, 1988).

Within a further embodiment, target cells are removed from the body, and a vector is introduced into the cells as a naked DNA plasmid. The transformed cells are then re-implanted into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, for example, Wu et al., J. Biol. Chem. 267:963-7, 1992; Wu et al., J. Biol. Chem. 263:14621-4, 1988.

Zkun6 proteins can also be used to prepare antibodies that specifically bind to zkun6 proteins. As used herein, the term “antibodies” includes polyclonal antibodies, monoclonal antibodies, antigen-binding fragments thereof such as F(ab′)₂ and Fab fragments, single chain antibodies, and the like, including genetically engineered antibodies. Non-human antibodies can be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. One skilled in the art can generate humanized antibodies with specific and different constant domains (i.e., different Ig subclasses) to facilitate or inhibit various immune functions associated with particular antibody constant domains. Alternative techniques for generating or selecting antibodies useful herein include in vitro exposure of lymphocytes to a zkun6 protein, and selection of antibody display libraries in phage or similar vectors (for instance, through use of immobilized or labeled zkun6 polypeptide). Antibodies are defined to be specifically binding if they bind to a zkun6 protein with an affinity at least 10-fold greater than the binding affinity to control (non-zkun6) polypeptide. It is preferred that the antibodies exhibit a binding affinity (Kₐ) of 10⁶ M⁻¹ or greater, preferably 10⁷ M⁻¹ or greater, more preferably 10⁸ M⁻ or greater, and most preferably 10⁹ M⁻¹ or greater. The affinity of a monoclonal antibody can be readily determined by one of ordinary skill in the art (see, for example, Scatchard, Ann. NYAcad. Sci. 51: 660-672, 1949).

Methods for preparing polyclonal and monoclonal antibodies are well known in the art (see for example, Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals such as horses, cows, goats, sheep, dogs, chickens, rabbits, mice, and rats. The immunogenicity of a zkun6 protein may be increased through the use of an adjuvant such as alum (aluminum hydroxide) or Freund's complete or incomplete adjuvant. Polypeptides useful for immunization also include fusion polypeptides, such as fusions of a zkun6 protein or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

Immunogenic zkun6 polypeptides may be as small as 5 residues. It is preferred to use polypeptides that are hydrophilic or comprise a hydrophilic region. Preferred such regions of SEQ ID NO:2 include residues 117-122, 525-530, 283-288, 50-55, and 402-407.

A variety of assays known to those skilled in the art can be utilized to detect antibodies that specifically bind to a zkun6 protein. Exemplary assays are described in detail in Antibodies: A Laboratory Manual, Harlow and Lane (Eds.), Cold Spring Harbor Laboratory Press, 1988. Representative examples of such assays include concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

Antibodies to zkun6 may be used for affinity purification of zkun6 proteins; within diagnostic assays for determining circulating levels of zkun6 proteins; for detecting or quantitating soluble zkun6 protein as a marker of underlying pathology or disease; for immunolocalization within whole animals or tissue sections, including immunodiagnostic applications; for immunohistochemistry; for screening expression libraries; and for other uses that will be evident to those skilled in the art. For certain applications, including in vitro and in vivo diagnostic uses, it is advantageous to employ labeled antibodies. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates.

Zkun6 proteins may be used in the laboratory or commercial preparation of proteins from cultured cells. The proteins can be used alone to inhibit specific proteolysis or can be combined with other proteinase inhibitors to provide a “cocktail” with a broad spectrum of activity. Of particular interest is the inhibition of cellular proteases, which can be released during cell lysis. Zkun6 proteins can also be used in the laboratory as a tissue culture additive to prevent cell detachment.

The invention is further illustrated by the following, non-limiting examples.

### EXAMPLE 1

A panel of cDNAs from human tissues was screened by PCR for zkun6 expression. The panel included 77 cDNA samples from various normal and cancerous human tissues and cell lines as shown in Table 5. The panel was set up in a 96-well format that included a human genomic DNA (Clontech Laboratories, Inc., Palo Alto, Calif.) positive control sample. Each well contained approximately 0.2-100 pg/μl of cDNA. The PCR reaction mixtures contained oligonucleotide primers ZC28,995 (SEQ ID NO:8) and ZC28,996 (SEQ ID NO:9), Taq DNA polymerase (ExTaq™; TAKARA Shuzo Co. Ltd., Biomedicals Group, Japan), and a density increasing agent and tracking dye (RediLoad™, Research Genetics, Inc., Huntsville, Ala.). The reaction mixtures were incubated at 94° C. for 2 minutes; followed by 35 cycles of 94° C. for 30 seconds, 61.4° C. for 30 seconds, and 72° C. for 30 seconds; followed by a 5-minute incubation at 72° C. About 10 μl of each of the PCR reaction products was electrophoresed on a 4% agarose gel. The predicted DNA fragment size of ˜110 bp was observed in brain, prostate, spinal cord, thyroid, fetal brain, placenta, salivary gland, testis, bone marrow, and stomach tumor, and possibly in islet, kidney, and HaCat cells.

The DNA fragments for brain, prostate, fetal brain, and genomic DNA were excised and purified using a commercially available gel extraction kit (obtained from Qiagen, Valencia, Calif.) according to the manufacturer's instructions. Fragments from fetal brain and genomic DNA were confirmed to be human zkun6 DNA by sequencing.

TABLE 5

| Tissue/Cell line | #Samples | Tissue/Cell line | #Samples |
|---|---|---|---|
| Adrenal gland | 1 | Bone marrow | 2 |
| Bladder | 1 | Fetal brain | 2 |
| Bone Marrow | 1 | Islet | 1 |
| Brain | 1 | Prostate | 2 |
| Cervix | 1 | RPMI #1788 | 2 |
| | | (ATCC CCL-156) | |
| Colon | 1 | Testis | 3 |
| Fetal brain | 1 | Thyroid | 1 |
| Fetal heart | 2 | WI38 (ATCC CCL-75) | 1 |
| Fetal kidney | 1 | Spinal cord | 1 |
| Fetal liver | 1 | HaCat (keratinocytes) | 1 |
| Fetal lung | 1 | HPV (prostate epitelia; | 1 |
| | | ATCC CRL-2221) | |
| Fetal muscle | 1 | MG63 (osteosarcoma) | 1 |
| Fetal skin | 1 | Prostate smooth muscle | 1 |
| Heart | 2 | CD3+ selected PBMC; | 1 |
| | | Ionomycin + PMA- | |
| | | stimulated | |
| K562 (keratinocyte; | 1 | HPVS (prostate epitelia, | 1 |
| ATCC CCL-243) | | selected; ATCC CRL-2221) | |
| Kidney | 1 | Heart | 1 |
| Liver | 1 | Pituitary | 1 |
| Lung | 1 | Placenta | 2 |
| Lymph node | 1 | Salivary gland | 1 |
| Melanoma | I | Mammary gland | 1 |
| Pancreas | 1 | Ovary | 1 |
| Pituitary | 1 | Adipocyte | 1 |
| Placenta | 1 | Prostate | 1 |
| Rectum | 1 | Salivary Gland | 1 |
| Small intestine | 1 | Skeletal muscle | 1 |
| Spinal cord | 1 | Spleen | 1 |
| Stomach | 1 | Testis | 2 |
| Thymus | 1 | Thyroid | 1 |
| Trachea | 1 | Uterus | 1 |
| Esophagus tumor | 1 | Stomach tumor | 1 |
| Liver tumor | 1 | Lung tumor | 1 |
| Ovarian tumor | 1 | Rectal tumor | 1 |
| Uterus tumor | 2 | | |

### EXAMPLE 2

Expressed sequence tags (ESTs) corresponding to the 5′ and 3′ ends of the human zkun6 sequence were obtained. Analysis of these ESTs and corresponding genomic sequence showed that there was a gap of approximately 270 bp between the 5′ and 3′ ESTs.

An arrayed fetal brain library (Example 1) was screened by PCR. This library represented 9.6×10⁵ clones in the vector pZP-9 (Example 4). A working plate containing 80 pools of 12,000 colonies each was screened by PCR for the presence of human zkun6 sequence. Screening was carried out using oligonucleotide primers ZC28,995 (SEQ ID NO:8) and ZC28,996 (SEQ ID NO:9) with an annealing temperature of 61.4° C. for 35 cycles. A second round of screening using oligonucleotide primers ZC29,898 (SEQ ID NO:10) and ZC29,899 (SEQ ID NO:11) with an annealing temperature of 76.0° C. for 35 cycles yielded one positive pool.

The second-round positive pool was plated and transferred to nylon membrane filters (Hybond-N™; Amersham Pharmacia Biotech, Piscataway, N.J.). Four filters at approximately 1000 colonies each were prepared. The filters were marked with a hot needle for orientation, then denatured for 6 minutes in 0.5 M NaOH and 1.5 M Tris-HCl pH 7.2. The filters were then neutralized in 1.5 M NaCl and 0.5 M Tris-HCl pH 7.2 for 6 minutes. The DNA was affixed to the filters using a UV crosslinker (Stratalinker®; Stratagene, La Jolla, Calif.) at 1200 joules. The filters were prewashed at 65° C. in prewash buffer (0.25× SSC, 0.25% SDS, 1 mM EDTA). The solution was changed a total of three times over a 45-minute period to remove cell debris. Filters were prehybridized overnight at 65° C. in 25 ml of a commercially available hybridization solution (Expresshyb™; Clontech Laboratories, Inc., Palo Alto, Calif..). A probe was generated by PCR using oligonucleotide primers ZC29,898 15 (SEQ ID NO:10) and ZC29,899 (SEQ ID NO:11), a positive clone from the fetal brain library as template, an annealing temperature of 76.0° C., and 35 cycles. The resulting PCR fragment was gel purified using a commercially available kit (QIAquick™ gel extraction kit; Qiagen). The probe was radioactively labeled with ³²p using a commercially available kit (Rediprime™ II random-prime labeling system; Amersham Pharmacia Biotech) according to the manufacturer's specifications. The probe was purified using a push column (NucTrap®; Stratagene Cloning Systems, La Jolla, Calif.). Hybridization took place overnight at 65° C. in a commercially available hybridization solution (Expresshyb™; Clontech Laboratories, Inc.). Filters were rinsed four times at 65° C. in pre-wash buffer, then exposed to film for 3 days at -80° C. There were 6 positives on the filters. Six clones were picked from the positive areas and streaked out. Ninety-five individual colonies from these six positives were screened by PCR using oligonucleotide primers ZC29,898 (SEQ ID NO:10) and ZC29,899 (SEQ ID NO:11) and an annealing temperature of 61.0° C. Two positives were obtained. One clone (designated clone #1) was sequenced and found to include the 3′ end and a sequence corresponding to the gap between the original ESTs.

To construct a full-length zkun6 cDNA, DNA was prepared from clone #1 and EST2906640 by the mini-prep method using a commercially available kit (obtained from Qiagen). A 1015-bp 5′-end fragment was generated by digesting EST2906640 with EcoRI and AatII. A 1085-bp 3′-end fragment was generated by digesting clone #1 with AatII and Xbal. The two fragments were ligated to plasmid pZP-9, which had been digested with EcoRI and XbaI. The ligation mixture was transformed into E. coli strain DH10B™ (obtained from Life Technologies, Inc., Gaithersburg, Md.) by electroporation. Ten clones were picked and checked by PCR using oligonucleotide primers ZC28,995 (SEQ ID NO:8) and ZC28,996 (SEQ ID NO:9) with an annealing temperature of 61.4° C. All clones were positive for the expected ˜110-bp band. One clone was sequenced and confirmed to encode human zkun6.

### EXAMPLE 3

A mouse expressed sequence tag (EST2278436) was found to include sequence corresponding to zkun6. The EST was sequenced and found to contain the 3′ coding region; it was missing ˜770 bp of the 5′ end.

11-day and 15-day mouse embryo cDNAs were screened for zkun6 by PCR using oligonucleotide primers ZC37,161 (SEQ ID NO:12) and ZC37,160 (SEQ ID NO:13) and Taq DNA polymerase (ExTaq™ DNA polymerase; TaKaRa Biomedicals) plus antibody. The reactions were run at an annealing temperature of 62.8° C. with an extension time of 30 seconds for a total of 35 cycles. Products of both reactions were positive.

The mouse 15-day embryo library was screened for a full-length clone.

This library was an arrayed library representing 9.6×10⁵ clones made in the vector pCMVSPORT2 (Life Technologies, Gaithersburg, Md.). A working plate containing 80 pools of 12,000 colonies each was screened by PCR using oligonucleotide primers ZC37,161 (SEQ ID NO:12) and ZC37,160 (SEQ ID NO:13) with an annealing temperature of 62.8° C. for 35 cycles. There were 3 positives. Pools corresponding to positive pools from the working plate were screened by PCR using the same reaction conditions. Four positives was obtained. Corresponding pools from the original source plates were then screened by PCR using the same reaction conditions. Reaction products were sequence and determined to represent mouse zkun6 DNA.

### EXAMPLE 4

A mammalian expression vector was constructed with the dihyrofolate reductase gene selectable marker under control of the SV40 early promoter, SV40 polyadenylation site, a cloning site to insert the gene of interest under control of the mouse metallothionein 1 (MT-1) promoter, and the human growth hormone (hGH) gene polyadenylation site. The expression vector was designated pZP-9 and has been deposited at the American Type Culture Collection, 10801 University Boulevard, Manassas, Va. under Accession No. 98668. To facilitate protein purification, the pZP-9 vector was modified by addition of a tissue plasminogen activator (t-PA) secretory signal sequence (see U.S. Pat. No. 5,641,655) and a Glu-Glu tag sequence (SEQ ID NO:4) between the MT-1 promoter and hGH terminator. The t-PA secretory signal sequence replaces the native secretory signal sequence for DNAs encoding polypeptides of interest that are inserted into this vector, and expression results in an N-terminally tagged protein. The N-terminally tagged vector was designated pZP9NEE.

To construct an expression vector for zkun6 or a portion thereof, PCR is performed on cDNA prepared as disclosed above. Primers are designed such that the PCR product will encode the desired polypeptide (e.g., an intact Kunitz domain or a multi-domain polypeptide) with restriction sites Bam HI in the sense primer and Xho I in the antisense primer to facilitate subcloning into an expression vector. 5 μl of 1/100 diluted cDNA, 20 pmoles of each oligonucleotide primer, and 1 U of a 2:1 mixture of ExTaq™ DNA polymerase (TaKaRa Biomedicals) and Pfu DNA polymerase (Stratagene, La Jolla, Calif.) (ExTaq/Pfu) are used in 25-μl reaction mixtures. The mixtures are incubated at 94° C. for 2 minutes; 3 cycles of 94° C. for 30 seconds, 50° C. for 30 seconds, 72° C. for 30 seconds; 35 cycles of 94° C. for 30 seconds, 68° C. for 30 seconds; and a 7-minute incubation at 72° C. The PCR product is gel purified and restriction digested with Bam HI and Xho I overnight. The vector pZPNEE is digested with Bam HI and Xho I, and the zkun6 fragment is inserted. The resulting construct is confirmed by sequencing.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An isolated protein comprising a portion of SEQ ID NO:2, wherein said portion is selected from the group consisting of residues 33-75, residues 93-157, residues 203-286, residues 299-351, and residues 412-548.

2. The isolated protein of claim 1 wherein said protein is from 43 to 1600 amino acid residues in length.

3. The isolated protein of claim 1 wherein the portion of SEQ ID NO:2 comprises residues 299-409 of SEQ ID NO:2.

4. The isolated protein of claim 1 wherein the portion of SEQ ID NO:2 comprises residues 33-548 of SEQ ID NO:2.

5. The isolated protein of claim 1 wherein the portion of SEQ ID NO:2 comprises residues 20-548 of SEQ ID NO:2.

6. The isolated protein of claim 1 further comprising an affinity tag.

7. An isolated protein comprising a portion of SEQ ID NO:2, wherein said portion is selected from the group consisting of residues 93-157, residues 203-286, residues 299-351, and residues 412-548.

8. An isolated polypeptide comprising at least 15 contiguous amino acid residues of SEQ ID NO:2, wherein the at least 15 contiguous amino acid residues comprise residues 117-122, 525-530, 283-288, or 50-55 of SEQ ID NO:2.

9. An expression vector comprising the following operably linked elements:
(a) a transcription promoter;
(b) a DNA segment encoding a protein comprising a portion of SEQ ID NO:2, wherein said portion is selected from the group consisting of residues 33-75, residues 93-157, residues 203-286, residues 299-351, and residues 412-548; and
(c) a transcription terminator.

10. The expression vector of claim 9 further comprising a secretory signal sequence operably linked to the DNA segment.

11. The expression vector of claim 10 wherein the secretory signal sequence encodes residues 1-19 of SEQ ID NO:2.

12. The expression vector of claim 9 wherein the portion of SEQ ID NO:2 comprises residues 299-409 of SEQ ID NO:2.

13. The expression vector of claim 9 wherein the portion of SEQ ID NO:2 comprises residues 33-548 of SEQ ID NO:2.

14. The expression vector of claim 9 wherein the portion of SEQ ID NO:2 comprises residues 20-548 of SEQ ID NO:2.

15. The expression vector of claim 9 wherein the vector further comprises a second DNA segment encoding an affinity tag operably linked to the DNA segment encoding the protein.

16. An expression vector comprising the following operably linked elements:
(a) a transcription promoter;
(b) a DNA segment encoding a protein comprising a portion of SEQ ID NO:2, wherein said portion is selected from the group consisting of residues 93-157, residues 203-286, residues 299-351, and residues 412-548; and
(c) a transcription terminator.

17. A cultured cell containing the expression vector of claim 9, wherein the cell expresses the DNA segment.

18. A cultured cell containing the expression vector of claim 16, wherein the cell expresses the DNA segment.

19. A method of making a protein comprising:
culturing the cell of claim 17 under conditions whereby the DNA segment is expressed; and
recovering the protein encoded by the DNA segment.

20. The method of claim 19 wherein the expression vector further comprises a secretory signal sequence operably linked to the DNA segment and wherein the protein is secreted into and recovered from a culture medium in which the cell is cultured.

21. A method of making a protein comprising:
culturing the cell of claim 18 under conditions whereby the DNA segment is expressed; and
recovering the protein encoded by the DNA segment.

22. The method of claim 21 wherein the expression vector further comprises a secretory signal sequence operably linked to the DNA segment and wherein the protein is secreted into and recovered from a culture medium in which the cell is cultured.

23. A protein produced by the method of claim 19.

24. A protein produced by the method of claim 21

25. An antibody that specifically binds to the protein of claim 1.


SEQUENCE LISTING (13 sequences)

SEQ ID NO: 1 - DNA, 2082 bp, Homo sapiens
  CDS (376)...(2022)
gtgaccctcatggccagtggctctgtgctcatgggcctctggcccctccccaacctcctcccctctgccctgtgctgaccagggcctgggagcccccgcacggttcagacagaggggccaggctgaagctggagaggaaccagcgtcacacagacggcctctgagaacttggagaccccgttacccacccagcaggggtgtcaggacaagcatctgctgcaggcttcagcctcaggggcaaaagggagccccggggtcctggtgggggcaccgaccacaggcccggagggtggatgcctgcaggaagctgggctctgtggagcccgaggaggggctggtggccacaccccccggccccctggctcggcggccctcatgcccgccctacgtccactcctgccgctcttgctcctcctccggctgacctcgggggctggcttgctgccagggctggggagccacccgggcgtgtgccccaaccagctcagccccaacctgtgggtggacgcccagagcacctgtgagcgcgagtgtagcagggaccaggactgtgcggctgctgagaagtgctgcatcaacgtgtgtggactgcacagctgcgtggcagcacgcttccccggcagcccagctgcgccgacgacagcggcctcctgcgagggctttgtgtgcccacagcagggctcggactgcgacatctgggacgggcagcccgtgtgccgctgccgcgaccgctgtgagaaggagcccagcttcacctgcgcctcggacggcctcacctactacaaccgctgctatatggacgccgaggcctgcctgcggggcctgcacctccacatcgtgccctgcaagcacgtgctcagctggccgcccagcagcccggggccgccggagaccactgcccgccccacacctggggccgcgcccgtgcctcctgccctgtacagcagcccctccccacaggcggtgcaggttgggggtacggccagcctccactgcgacgtcagcggccgcccgccgcctgctgtgacctgggagaagcagagtcaccagcgagagaacctgatcatgcgccctgatcagatgtatggcaacgtggtggtcaccagcatcgggcagctggtgctctacaacgcgcggcccgaagacgccggcctgtacacctgcaccgcgcgcaacgctgctgggctgctgcgggctgacttcccactctctgtggtccagcgagagccggccagggacgcagcccccagcatcccagccccggccgagtgcctgccggatgtgcaggcctgcacgggccccacttccccacaccttgtcctctggcactacgacccgcagcggggcggctgcatgaccttcccggcccgtggctgtgatggggcggcccgcggctttgagacctacgaggcatgccagcaggcctgtgcccgcggccccggcgacgcctgcgtgctgcctgccgtgcagggcccctgccggggctgggagccgcgctgggcctacagcccgctgctgcagcagtgccatcccttcgtgtacggtggctgcgagggcaacggcaacaacttccacagccgcgagagctgcgaggatgcctgccccgtgccgcgcacaccgccctgccgcgcctgccgcctccggagcaagctggcgctgagcctgtgccgcagcgacttcgccatcgtggggcggctcacggaggtgctggaggagcccgaggccgccggcggcatcgcccgcgtggcgctcgaggacgtgctcaaggatgacaagatgggcctcaagttcttgggcaccaagtacctggaggtgacgctgagtggcatggactgggcctgcccctgccccaacatgacggcgggcgacgggccgctggtcatcatgggtgaggtgcgcgatggcgtggccgtgctggacgccggcagctacgtccgcgccgccagcgagaagcgcgtcaagaagatcttggagctgctggagaagcaggcctgcgagctgctcaaccgcttccaggactagcccccgcaggggcctgcgccaccccgtcctggtgaataaacgcactccctgtgcctcaga

SEQ ID NO: 2 - protein, 548 aa, Homo sapiens
MPALRPLLPLLLLLRLTSGAGLLPGLGSHPGVCPNQLSPNLWVDAQSTCERECSRDQDCAAAEKCCINVCGLHSCVAARFPGSPAAPTTAASCEGFVCPQQGSDCDIWDGQPVCRCRDRCEKEPSFTCASDGLTYYNRCYMDAEACLRGLHLHIVPCKHVLSWPPSSPGPPETTARPTPGAAPVPPALYSSPSPQAVQVGGTASLHCDVSGRPPPAVTWEKQSHQRENLIMRPDQMYGNVVVTSIGQLVLYNARPEDAGLYTCTARNAAGLLRADFPLSVVQREPARDAAPSIPAPAECLPDVQACTGPTSPHLVLWHYDPQRGGCMTFPARGCDGAARGFETYEACQQACARGPGDACVLPAVQGPCRGWEPRWAYSPLLQQCHPFVYGGCEGNGNNFHSRESCEDACPVPRTPPCRACRLRSKLALSLCRSDFAIVGRLTEVLEEPEAAGGIARVALEDVLKDDKMGLKFLGTKYLEVTLSGMDWACPCPNMTAGDGPLVIMGEVRDGVAVLDAGSYVRAASEKRVKKILELLEKQACELLNRFQD

SEQ ID NO: 3 - protein, 55 aa, Homo sapiens
TDICKLPKDEGTCRDFILKWYYDPNTKSCARFWYGGCGGNENKFGSQKECEKVCA

SEQ ID NO: 4 - protein, 6 aa, Artificial Sequence
  Glu-Glu tag
EYMPME

SEQ ID NO: 5 - protein, 55 aa, Artificial Sequence
  peptide motif
CXXXXXXXXCXXXXXXXXXXXXXXXXXXXCXXXXXXXCXXXXXXXXXXXXCXXXC

SEQ ID NO: 6 - protein, 4 aa, Artificial Sequence
  thrombin cleavage site
LVPR

SEQ ID NO: 7 - DNA, 1644 bp, Artificial Sequence
  degenerate sequence
atgccngcnytnmgnccnytnytnccnytnytnytnytnytnmgnytnacnwsnggngcnggnytnytnccnggnytnggnwsncayccnggngtntgyccnaaycarytnwsnccnaayytntgggtngaygcncarwsnacntgygarmgngartgywsnmgngaycargaytgygcngcngcngaraartgytgyathaaygtntgyggnytncaywsntgygtngcngcnmgnttyccnggnwsnccngcngcnccnacnacngcngcnwsntgygarggnttygtntgyccncarcarggnwsngaytgygayathtgggayggncarccngtntgymgntgymgngaymgntgygaraargarccnwsnttyacntgygcnwsngayggnytnacntaytayaaymgntgytayatggaygcngargcntgyytnmgnggnytncayytncayathgtnccntgyaarcaygtnytnwsntggccnccnwsnwsnccnggnccnccngaracnacngcnmgnccnacnccnggngcngcnccngtnccnccngcnytntaywsnwsnccnwsnccncargcngtncargtnggnggnacngcnwsnytncaytgygaygtnwsnggnmgnccnccnccngcngtnacntgggaraarcarwsncaycarmgngaraayytnathatgmgnccngaycaratgtayggnaaygtngtngtnacnwsnathggncarytngtnytntayaaygcnmgnccngargaygcnggnytntayacntgyacngcnmgnaaygcngcnggnytnytnmgngcngayttyccnytnwsngtngtncarmgngarccngcnmgngaygcngcnccnwsnathccngcnccngcngartgyytnccngaygtncargcntgyacnggnccnacnwsnccncayytngtnytntggcaytaygayccncarmgnggnggntgyatgacnttyccngcnmgnggntgygayggngcngcnmgnggnttygaracntaygargcntgycarcargcntgygcnmgnggnccnggngaygcntgygtnytnccngcngtncarggnccntgymgnggntgggarccnmgntgggcntaywsnccnytnytncarcartgycayccnttygtntayggnggntgygarggnaayggnaayaayttycaywsnmgngarwsntgygargaygcntgyccngtnccnmgnacnccnccntgymgngcntgymgnytnmgnwsnaarytngcnytnwsnytntgymgnwsngayttygcnathgtnggnmgnytnacngargtnytngargarccngargcngcnggnggnathgcnmgngtngcnytngargaygtnytnaargaygayaaratgggnytnaarttyytnggnacnaartayytngargtnacnytnwsnggnatggaytgggcntgyccntgyccnaayatgacngcnggngayggnccnytngtnathatgggngargtnmgngayggngtngcngtnytngaygcnggnwsntaygtnmgngcngcnwsngaraarmgngtnaaraarathytngarytnytngaraarcargcntgygarytnytnaaymgnttycargay

SEQ ID NO: 8 - DNA, 22 bp, Artificial Sequence
  oligonucleotide primer ZC28,995
acttccccacaccttgtcctct

SEQ ID NO: 9 - DNA, 21 bp, Artificial Sequence
  oligonucleotide primer ZC28,996
tgcctcgtaggtctcaaagcc

SEQ ID NO: 10 - DNA, 23 bp, Artificial Sequence
  oligonucleotide primer ZC29,898
gtcctctggcactacgacccgca

SEQ ID NO: 11 - DNA, 19 bp, Artificial Sequence
  oligonucleotide primer ZC29,899
acggcaggcagcacgcagg

SEQ ID NO: 12 - DNA, 22 bp, Artificial Sequence
  oligonucleotide primer ZC37,161
cctgaccaaatgtatggcaacg

SEQ ID NO: 13 - DNA, 23 bp, Artificial Sequence
  oligonucleotide primer ZC37,160
cctgggtccctgtcctgagtagt